SEQUENCE LISTING (11 sequences)

SEQ ID NO: 1 - protein, 477 aa, Mus musculus
MSAAPGLLRQELSCPLCLQLFDAPVTAECGHSFCRACLIRVAGEPAADGTVACPCCQAPTRPQALSTNLQLSRLVEGLAQVPQGHCEEHLDPLSIYCEQDRTLVCGVCASLGSHRGHRLLPAAEAQARLKTQLPQQKMQLQEACMRKEKTVAVLEHQLVEVEETVRQFRGAVGEQLGKMRMFLAALESSLDREAERVRGDAGVALRRELSSLNSYLEQLRQMEKVLEEVADKPQTEFLMKFCLVTSRLQKILSESPPPARLDIQLPVISDDFKFQVWKKMFRALMPALEELTFDPSSAHPSLVVSSSGRRVECSDQKAPPAGEDTRQFDKAVAVVAQQLLSQGEHYWEVEVGDKPRWALGVMAADASRRGRLHAVPSQGLWLLGLRDGKILEAHVEAKEPRALRTPERPPARIGLYLSFADGVLAFYDASNPDVLTPIFSFHERLPGPVYPIFDVCWHDKGKNAQPLLLVGPEQEQA

SEQ ID NO: 2 - protein, 477 aa, Homo sapiens
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 3 - protein, 43 aa, synthetic construct
  REGION 1..43: note = human TRIM72 Ring-finger domain 14-56 aa
CPLCLQLFDAPVTAECGHSFCRACLGRVAGEPAADGTVLCPCC

SEQ ID NO: 4 - protein, 32 aa, synthetic construct
  REGION 1..32: note = human TRIM72 B-box domain 86-117aa)
CEEHLDPLSIYCEQDRALVCGVCASLGSHRGH

SEQ ID NO: 5 - protein, 98 aa, synthetic construct
  REGION 1..98: note = human TRIM72 coiled-coil domain (135-232 aa)
QQKLQLQEACMRKEKSVAVLEHQLVEVEETVRQFRGAVGEQLGKMRVFLAALEGSLDREAERVRGEAGVALRRELGSLNSYLEQLRQMEKVLEEVADK

SEQ ID NO: 6 - protein, 193 aa, synthetic construct
  REGION 1..193: note = human TRIM72 PRYSPRY domain (278-470 aa)
RKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLV

SEQ ID NO: 7 - protein, 421 aa, synthetic construct
  REGION 1..421: note = TRIM72 Ring domain (deletion of 14-69aa)
MSAAPGLLHQELSQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 8 - protein, 435 aa, synthetic construct
  REGION 1..435: note = TRIM72 B-box domain (deletion of 81-122aa)
MSAAPGLLHQELSCPLCLQLFDAPVTAECGHSFCRACLGRVAGEPAADGTVLCPCCQAPTRPQALSTNLQLARLVEGLAQAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 9 - protein, 379 aa, synthetic construct
  REGION 1..379: note = TRIM72 coiled-coil domain (deletion of 135-232aa)
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLVGPEGAEA

SEQ ID NO: 10 - protein, 284 aa, synthetic construct
  REGION 1..284: note = TRIM72 PRYSPRY domain (deletion of 278-470aa)
MSAAPGLLHQELSCPLCLQLFDAPVTAECGHSFCRACLGRVAGEPAADGTVLCPCCQAPTRPQALSTNLQLARLVEGLAQVPQGHCEEHLDPLSIYCEQDRALVCGVCASLGSHRGHRLLPAAEAHARLKTQLPQQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWGPEGAEA

SEQ ID NO: 11 - protein, 336 aa, synthetic construct
  REGION 1..336: note = TRIM72 coiled-coil domain plus PRYSPRY domain (135-470aa)
QQKLQLQEACMRKEKSVAVLEHQLVEVEETVRQFRGAVGEQLGKMRVFLAALEGSLDREAERVRGEAGVALRRELGSLNSYLEQLRQMEKVLEEVADKPQTEFLMKYCLVTSRLQKILAESPPPARLDIQLPIISDDFKFQVWRKMFRALMPALEELTFDPSSAHPSLVVSSSGRRVECSEQKAPPAGEDPRQFDKAVAVVAHQQLSEGEHYWEVDVGDKPRWALGVIAAEAPRRGRLHAVPSQGLWLLGLREGKILEAHVEAKEPRALRSPERRPTRIGLYLSFGDGVLSFYDASDADALVPLFAFHERLPRPVYPFFDVCWHDKGKNAQPLLLV

(19) United States
(12) Patent Application Publication
(10) Pub. No.: US 2025/0382342 A1
(43) Pub. Date: Dec. 18, 2025

(54) A TRUNCATED PROTEIN AND USE THEREOF

(71) Applicant: SINEUGENE THERAPEUTICS CO., LTD., Beijing (CN)

(72) Inventors: Yichang JIA, Beijing (CN); Wei GUO, Beijing (CN); Lin PENG, Beijing (CN)

(21) Appl. No.: 18/879,150

(22) PCT Filed: Jun. 27, 2023
(86) PCT No.: PCT/CN2023/102720

§ 371 Date: Dec. 26, 2024

(30) Foreign Application Priority Data
Jun. 28, 2022 (WO) PCT/CN2022/101918

(51) Int. Cl. C07K 14/47; A61K 38/00; A61P 25/16; C12N 15/62; C12N 15/864

(52) U.S. Cl. CPC C07K 14/4716; A61K 38/00; A61P 25/16; C12N 15/62; C12N 15/8645; C07K 2319/21; C07K 2319/73; C07K 2319/95; C12N 2750/14143

(57) ABSTRACT

Provided is a TRIM72 truncated protein, comprising the Coiled-PRYSPRY domain or PRYSPRY domain or its functional fragment of a TRIM72 protein. Further provided is the use of the TRIM72 truncated protein.

### BACKGROUND OF THE INVENTION

Amyotrophic lateral sclerosis (ALS), also known as Lou Gehrig's disease and motor neuron disease (MND), is a specific disease that causes the death of neurons which control voluntary muscles. Some also use the term “motor neuron disease” for a group of conditions of which ALS is the most common. ALS is characterized by stiff muscles, muscle twitching, and gradually worsening weakness due to muscles decreasing in size. This results in difficulty in speaking, swallowing, and eventually breathing. The cause is not known in 90% to 95% of case. About 5-10% of cases are inherited from a person's parents. About half of theses genetic cases are due to four specific genes, SOD1, TDP-43, FUS, and C9orf72. No cure for ALS is known at present.

TRIM72 is a Tripartite Motif (TRIM) family protein that consists of a Ring finger, a B-box motif, a coiled-coil region and a C-terminal PRYSPRY domain. It participates in sarcolemmal membrane repair process and is associated with insulin signaling pathway. It also takes part in cardioprotection against Ischemia/Reperfusion (IR) injury. Full-length TRIM72 work as a potential target for ALS through ubiquitinating mutant FUS protein has been reported. However, the delivery of the full-length TRIM72 is greatly limited by the characteristic of the vector, some vector has limited size of delivered genes. Therefore, the effect of TRIM72 for protecting neurons needed to be further explored, and different TRIM72 protein product needs to be further developed.

### SUMMARY OF THE INVENTION

The present disclosure provides a TRIM72 truncated protein and use thereof. The TRIM72 truncated protein has one or more of the following properties: (1) capable of protecting neurons effectively; (2) capable of reducing oxidative stress; (3) capable of treating, preventing and/or alleviating nervous system disease.

In one aspect, the present application provides a TRIM72 truncated protein, comprising the PRYSPRY domain or its functional fragment of a TRIM72 protein.

In some embodiments, the TRIM72 protein is a human TRIM72 protein.

In some embodiments, the TRIM72 protein comprises an amino acid sequence of SEQ ID NO: 2.

In some embodiments, the PRYSPRY domain comprises amino acid sites of 278aa-470aa of the TRIM72 protein.

In some embodiments, the PRYSPRY domain comprises an amino acid sequence as set forth in SEQ ID NO: 6.

In some embodiments, the TRIM72 truncated protein further comprising the coiled-coil domain or its functional fragment of a TRIM72 protein.

In some embodiments, the TRIM72 truncated protein does not comprise the coiled-coil domain or its functional fragment of a TRIM72 protein.

In some embodiments, the coiled-coil domain comprises amino acid sites of 135aa-232aa of the TRIM72 protein.

In some embodiments, the coiled-coil domain comprises an amino acid sequence as set forth in SEQ ID NO: 5.

In some embodiments, the TRIM72 truncated protein further comprising the B-box domain or its functional fragment of a TRIM 72 protein.

In some embodiments, the TRIM72 truncated protein does not comprise the B-box domain or its functional fragment of a TRIM72 protein.

In some embodiments, the B-box domain comprises amino acid sites of 86aa-117aa of the TRIM72 protein.

In some embodiments, the B-box domain comprises an amino acid sequence as set forth in SEQ ID NO: 4.

In some embodiments, the TRIM72 truncated protein further comprising the Ring-finger domain or its functional fragment of a TRIM 72 protein.

In some embodiments, the TRIM72 truncated protein does not comprise the Ring-finger domain or its functional fragment of a TRIM72 protein.

In some embodiments, the Ring-finger domain comprises amino acid sites of 14aa-56aa of the TRIM72 protein.

In some embodiments, the Ring-finger domain comprises an amino acid sequence as set forth in SEQ ID NO: 3.

In some embodiments, the TRIM72 truncated protein comprises an amino acid sequence as set forth in any one of SEQ ID NO: 6, 7, 8, 9 and 11.

In some embodiments, the TRIM72 truncated protein comprises its variant thereof.

In some embodiments, the TRIM72 truncated protein does not comprise an amino acid mutation at position C242.

In some embodiments, the TRIM72 truncated protein further comprises an amino acid mutation at position C14.

In some embodiments, the TRIM72 truncated protein further comprises an amino acid mutation C14A.

In some embodiments, the TRIM72 truncated protein is used for protect neurons by reducing oxidative stress.

In some embodiments, the TRIM72 truncated protein is secreted through exosome.

In some embodiments, the TRIM72 truncated protein is used for preventing or treating a nervous system disease.

In some embodiments, the nervous system disease is a nerve damage disease induced by oxidative stress.

In some embodiments, the TRIM72 truncated protein is used for preventing or treating ALS, PD and/or Stroke.

In another aspect, the present application provides a recombinant protein, comprising the TRIM72 truncated protein.

In another aspect, the present application provides one or more isolated nucleic acid molecules, encoding the TRIM72 truncated protein.

In another aspect, the present application provides a vector, comprising the nucleic acid molecule.

In some embodiments, the vector comprises a viral vector.

In some embodiments, the vector comprises an AAV vector.

In some embodiments, the viral vector comprises an AAV9 vector.

In another aspect, the present application provides a cell, comprising the nucleic acid molecule, or the vector.

In another aspect, the present application provides a fusion protein, comprising the TRIM72 truncated protein.

In another aspect, the present application provides a pharmaceutical composition, comprising said TRIM72 truncated protein, the recombinant protein, the nucleic acid molecule, the vector, the cell and/or the fusion protein, and a pharmaceutically accepted adjuvant.

In some embodiments, the pharmaceutically accepted adjuvant comprises drug, toxins, cytokines, radioactive elements, carrier proteins, enzymes, lectins, fluorescent quantum dots, and/or high absorption coefficient of chromophore.

In another aspect, the present application provides a method for protecting neurons in a subject, comprising administering an effective amount of the TRIM72 truncated protein, the recombinant protein, the nucleic acid molecule, the vector, said cell, the fusion protein, and/or the pharmaceutical composition to a subject in need thereof.

In another aspect, the present application provides a method for preventing and/or treating a nervous system disease, comprising administering an effective amount of the TRIM72 truncated protein, the recombinant protein, the nucleic acid molecule, the vector, said cell, the fusion protein, and/or the pharmaceutical composition to a subject in need thereof.

In some embodiments, the nervous system disease is a nerve damage disease induced by oxidative stress,

In some embodiments, the nervous system disease is a neurodegenerative disease.

In some embodiments, the nervous system disease comprising ALS, PD, and/or ALS.

In another aspect, the present application provides a use of the TRIM72 truncated protein, the recombinant protein, the nucleic acid molecule, the vector, said cell, the fusion protein, and/or the pharmaceutical composition in manufacture of a drug for preventing and/or treating a nervous system disease.

In some embodiments, the nervous system disease is a nerve damage disease induced by oxidative stress,

In some embodiments, the nervous system disease is a neurodegenerative disease.

In some embodiments, the nervous system disease comprising ALS, PD, and/or Stroke.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates loss-of-function of TRIM72 accelerates disease progression shown in FUS-R521C knockin mouse. (A) TRIM72 was upregulated in C/C but not in +/+ cultured mouse motor neurons (MNs, 3 DIV). RT-PCR, 3 biological replicates, Gapdh as loading control. (B and C) The number of ChAT-positive motor neuron in the ventral horns at 1 year of age (n≥3). The ChAT-positive motor neurons in sections from lumbar 4-5 spinal cords. Scale bar in B, 50 μm. (D) Survival curve of indicated genotypes. Loss-of-function of TRIM72 significantly shortened the life span of the FUS-R521C knockin mice. Mice, male, n=25 (+/+), 19 (C/C), 17 (C/C;−/−), and 18 (−/−).

FIG. 2. Illustrates loss-of-function of TRIM72 increases oxidative stress. (A) The present cortical neuron of DHE (Dihydroethidium) and MitoSOX staining in the indicated genotypes. DIV12-14. (B, C) Both DHE and MitoSOX staining supported higher oxidative stress in C/C;−/− cortical cultures than other three genotypes. The cells treated with AS and H₂O₂ were served as positive controls for oxidative stress. The values are presented as mean±SEM (n=3-4). **p<0.01 (ANOVA, SPSS). N.S., no statistical significance (ANOVA, SPSS).

FIG. 3 illustrates TRIM72 protects cell against oxidative stress. (A) TRIM72 domain annotation and the key residues for TRIM72 previously reported functions were labeled. (B) Expression of Flag-tagged domain-disrupt TRIM72 fused with EGFP. GAPDH served as protein loading control. FL, full-length TRIM72. (C) Domain-disrupt of TRIM72 on cell viability after stress challenge. After AS treatment, cell viability/dehydrogenase activity was measured by WST-8 assay within two hours. The expression of TRIM72 protected the cell from AS-induced stress and increased cell viability. The Coiled-coil domain- or PRYSPRY domain-disrupt not Ring domain- or B-box domain-disrupt abolished the protective effect of TRIM72. (D) Expression of the TRIM72 mutants on cell viability after stress challenge. The C242A but not C14A mutation abolished the anti-stress effect of TRIM72. The values are presented as mean±SEM. In C and D, the values were generated from at least three independent experiments (n≥3). *p<0.05, **p<0.01, ***p<0.001 (t-test or ANOVA, SPSS). N.S., no statistical significance.

FIG. 4 illustrates TRIM72 protects cell against oxidative stress in cultured neurons. (A) The DHE staining of cortical neuron after expression of TRIM72 by lentivirus infection. The cortical neuron isolated from C/C;−/− mice (P0, DIV12), Scale bar, 20 μm. (B) The mean intensity of DHE was reduced by expression of TRIM72. The images were analyzed by Fiji ImageJ. the mean DHE fluorescent intensity was measured, substrate background. The values are presented as mean±SEM. ***p<0.001 (t-test).

FIG. 5 illustrates reduction of ROS level by treatment of TRIM72 protein and its fragments. (A) Schematic diagram showing TRIM72 protein and its fragments purified from E. coli. (B) The DHE staining of cortical neuron after treatment of TRIM72 proteins at the concentration of 0, 10, 20 or 40 μg/ml. The values are presented as mean±SEM. ***p<0.001 (ANOVA, SPSS). N.S., no statistical significance.

FIG. 6 illustrates cell viability under oxidative stress. (A) Schematic diagram showing full-length and different-truncated TRIM72 constructs in pAAV-ITR vectors. (B) The cell viability under H₂O₂ stress with expression of full length or different-truncated form of TRIM72 by scAAV9 infection. The values are presented as mean±SEM with all data points and t-test was performed to compare the datasets with control. **p<0.01 *p<0.05, N.S., no statistical significance.

FIG. 7 illustrates the expression pattern of TRIM72 in exosomes by western blot analysis.

FIG. 8 illustrates the secretion efficiency of full-length or different-truncated TRIM72 in exosomes. The values are presented as mean±SEM with all data points. One-way ANOVA was performed to compare the datasets, ***p<0.001, N.S., no statistical significance.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “TRIM72 protein” can be used interchangeably with “MG53” protein, generally include a TRIM72 protein or its variant, functional fragment, analogue, homologue. The TRIM72 protein may contain a Ring finger, a B-box motif, a coiled-coil region and/or a C-terminal PRYSPRY domain. For example, the Ring-finger domain may comprise amino acid sites of 14aa-56aa of the TRIM72 protein or its functional fragment. For example, the B-box domain may comprise amino acid sites of 86aa-117aa of the TRIM72 protein or its functional fragment. For example, the coiled-coil domain may comprise amino acid sites of 135aa-232aa of the TRIM72 protein or its functional fragment. For example, the PRYSPRY domain may comprise amino acid sites of 278aa-470aa of the TRIM72 protein. The term may also include the TRIM72 protein derived from any known species which has a TRIM72 protein.

In the present application, the term “truncated protein” generally refers to a protein with one or more amino acid deletion compared with the full-length protein. For example, the truncated protein may contain the main functional fragment of the protein. For example, the truncated protein also includes but not limited to its variant, functional fragment, analogue, homologue.

In the present application, the “amino acid mutation Xn” refers to an amino acid mutation occurring in the amino acid residue X at position n of the amino acid sequence as set forth in SEQ ID NO: 2, wherein n is a positive integer, X is an abbreviation of any amino acid residue. For example, the “amino acid mutation C14” refers to the amino acid substitution occurring in the amino acid residue C corresponding to position 14 of the amino acid sequence as set forth in SEQ ID NO: 2.

The amino acid mutations of the present application can be non-conserved mutations. Said non-conserved mutations can comprise changing the amino acid residues in a target protein or polypeptide in a non-conserved manner, e.g., replacing an amino acid residue having a certain side chain size or a certain characteristic (e.g., hydrophilic) with an amino acid residue having a different side chain size or a different characteristic (e.g., hydrophobic).

Said amino acid substitutions can also be conserved substitutions. Said conserved substitutions can comprise changing the amino acid residues in a target protein or polypeptide in a conserved manner, e.g., replacing an amino acid residue having a certain side chain size or a certain characteristic (e.g., hydrophilic) with an amino acid residue having the same or similar side chain size or the same or similar characteristic (e.g., still hydrophilic). Such conserved substitutions generally would not produce a significant effect on the structure or the function of the produced protein. In the present application, the amino acid sequence variant which is a mutant of the fusion protein, its fragment, or its variant which undergoes one or more amino acid substitutions can comprise conserved amino acid substitutions that would not remarkably change the structure or function of the protein.

As an example, the mutual substitutions between amino acids in each of the following groups can be considered as conservative substitutions in the present application:
Group of amino acids with nonpolar side side(s): alanine, valine, leucine, isoleucine, proline, phenylalanine, tryptophan and methionine.
Group of uncharged amino acids with polar side chains: glycine, serine, threonine, cysteine, tyrosine, asparagine and glutamine.
Group of negatively charged amino acids with polar side chains: aspartic acid and glutamic acid.
Group of positively charged basic amino acids: lysine, arginine and histidine.
Group of amino acids with phenyl: phenylalanine, tryptophan and tyrosine.

In the present application, the term “fusion protein” generally refers to a complex polypeptide, that is, a single continuous amino acid sequence consisting of two (or more) polypeptides. The fusion protein can generally be artificially prepared by means of recombinant nucleic acid or chemical synthesis.

In the present application, the term “neurodegenerative disease” generally refers to a varied assortment of nervous system disorders characterized by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue. For example, the neurodegenerative disease can be caused by oxidative stress.

In the present application, the term “Parkinson's disease” generally refers to a kind of neurodegenerative disorder. The Parkinson's disease is a chronic progressive nervous disease characterised by neurodegeneration, especially degeneration of dopaminergic neurons. Symptoms include stooped posture, resting tremor, weakness of resting muscles, a shuffling gait, speech impediments, movement difficulties and an eventual slowing of mental processes and/or dementia.

In the present application, the term “stroke” generally refers to conditions caused by the occlusion or hemorrhage of one or more blood vessels supplying the brain, which may lead to cell death. The term can include ischemic stroke and hemorrhagic stroke. “Ischemic stroke”, as used herein, generally refers to stroke caused by an occlusion of one or more blood vessels supplying the brain. Types of ischemic stroke can include but not limited to, e.g., embolic stroke, cardioembolic stroke, thrombotic stroke, large vessel thrombosis, lacunar infarction, artery-artery stroke and cryptogenic stroke. “Hemorrhagic stroke”, as used herein, generally refers to stroke caused by hemorrhage of one or more blood vessels supplying the brain. Types of hemorrhagic stroke include, e.g., subdural stroke, intraparenchymal stroke, epidural stroke and subarachnoid stroke.

In the present application, the term “nucleic acid molecule” generally refers to an isolated form of nucleotide, deoxyribonucleotide or ribonucleotide or their analogs of any length isolated from their natural environment or artificially synthesized. The nucleic acid molecules of the present application can be isolated. For example, it can be produced or synthesized by the following ways: (i) in vitro amplification, such as polymerase chain reaction (PCR) amplification, (ii) clonal recombination, (iii) purification, e.g., fractionation by restriction enzyme digestion and gel electrophoresis, or (iv) synthesis, e.g., chemical synthesis. In some embodiments, said isolated nucleic acid is a nucleic acid molecule prepared by a recombinant DNA technology. In the present application, the nucleic acid encoding said truncated protein or its functional fragment can be prepared by a variety of methods known in the art. These methods include, but are not limited to, overlap extension PCR by use of restriction fragment operations or synthetic oligonucleotides. Specific operations can be found in Sambrook et al., Molecular Cloning, A Laboratory Manual, Cold Spring Harbor Laboratory Press, Cold Spring Harbor, NY, 1989; and Ausube et al. Current Protocols in Molecular Biology, Greene Publishing and Wiley-Interscience, New York NY, 1993.

In the present application, the term “vector” generally refers to a vector containing a recombinant polynucleotide, where the recombinant polynucleotide includes an expression control sequence efficiently linked to a nucleotide sequence to be expressed. The vector includes cis-acting elements sufficient for expression; other elements for expression may be provided by the host cell or may be provided in an in-vitro expression system. The vector may include all expression vectors known in the art that can be incorporated into the recombinant polynucleotide, including cosmid, plasmid (e.g., naked or encapsulated in liposomes) and viruses (e.g., lentiviruses, retroviruses, adenoviruses, and adeno-associated viruses).

In the present application, the term “encoding” generally refers to the inherent property of a particular sequence of nucleotides in a polynucleotide such as a gene, cDNA or mRNA to act as a template for the synthesis of other multimers and macromolecules in a biological process, said multimers and macromolecules having either a defined sequence of nucleotides (i.e., rRNA, tRNA and mRNA) or a defined sequence of amino acids and the biological properties arising therefrom. Thus, if transcription and translation of an mRNA corresponding to a gene produces a protein in a cell or other biological system, the gene encodes the protein. Both the coding strand whose nucleotide sequence is identical to the mRNA sequence and is usually provided in the sequence listing, and the non-coding strand used as a template for the transcription of a gene or cDNA may be referred to as the protein or other product encoding the gene or cDNA. In the present application, the term “coding element” generally refers to a nucleic acid (an RNA or DNA molecule) including a nucleotide sequence encoding a protein.

In the present application, the terms “host cell”, “cell”, and “host” are used interchangeably, and generally refer to a plasmid or vector that can include or have included the nucleic acid molecule of the present application, or can express individual cells, cell lines or cell cultures of the protein of the present application, its fragments or its variants. Said host cell can comprise the progeny of a single host cell. Due to natural, accidental or deliberate mutations, the progeny cells and the original parent cells can not necessarily be completely identical in morphology or genome, as long as they can express the protein of the present application or its fragments. Said host cell can be obtained by transfecting cells in vitro with the vector of the present application. Said host cell can be a prokaryotic cell (e.g., Escherichia coli) or a eukaryotic cell (e.g., yeast cells, e.g., COS cells, Chinese Hamster Ovary (CHO) cells, HeLa cells, HEK293 cells, COS-1 cells, NS0 cells or myeloma cells). In the present application, said host cell can be a CHO cell.

In the present application, the term “treat” generally refers to slowing or improving the progression, severity, and/or duration of a proliferative condition, or improving one or more symptoms (e.g., one or more distinguishable symptoms) of a proliferative condition as a result of the administration of one or more therapies.

In the present application, the term “subject” generally refers to any human or non-human animal. The term “non-human animal” can include all vertebrates, such as, mammals and non-mammals, e.g., non-human primates, goats, sheep, dogs, cows, chickens, amphibians, reptiles, etc.

In the present application, the terms “peptide”, “polypeptide” and “protein” can be used interchangeably and generally refer to compounds composed of amino acid residues covalently linked by peptide bonds. The protein or peptide must contain at least two amino acids, and there is no limitation on the maximum number of amino acids that can be included in the protein or peptide sequence. The polypeptide may include any peptides or proteins that contain two or more amino acids linked to each other through peptide bonds. In the present application, this term refers to two short chains, which are also commonly known as peptides, oligopeptides and oligomers in the art, for example longs chains, which are commonly known as proteins in the art, of which there are many types. “Polypeptides” include, for example, bioactive fragments, substantially homologous polypeptides, oligopeptides, homodimers, heterodimers, variants of polypeptides, modified polypeptides, derivatives, analogues, fusion proteins, etc. Polypeptides include native peptides, recombinant peptides or combinations thereof.

In addition to particular proteins and nucleotides mentioned herein, the present application may also include their functional variants, derivatives, analogues, homologues and fragments thereof.

The term “functional variant” refers to a polypeptide having substantially the same amino acid sequence or encoded by substantially the same nucleotide sequence as the naturally occurring sequence and capable of having one or more activities of the naturally occurring sequence. In the context of the present application, the variant of any given sequence refers to a sequence in which a particular sequence of residues (either amino acid or nucleotide residues) has been modified so that the polypeptide or polynucleotide remains substantially at least one endogenous function. The variant sequences can be obtained through the addition, deletion, substitution, modification, replacement and/or variation of at least one amino acid residue and/or nucleotide residue present in a naturally occurring protein and/or polynucleotide, as long as the original functional activity is maintained. In the present application, the term “derivative” generally refers to a polypeptide or polynucleotide of the present application including any substitution, variation, modification, replacement, deletion and/or addition from/to one (or more) amino acid residues of the sequence, provided that the resulting polypeptide or polynucleotide substantially maintains at least one of its endogenous functions.

In the present application, the term “analogue” generally, with respect to a polypeptide or polynucleotide, includes any mimetic of the polypeptide or polynucleotide, that is, a chemical compound having at least one endogenous function of the polypeptide or polynucleotide that the mimetic mimics. In general, amino acids can be substituted, for example, at least 1 (e.g., 1, 2, 3, 4, 5, 6, 7, 8, 9, 10 or 20 or above) amino acids can be substituted, provided that the modified sequence substantially maintains the required activity or capability. Amino acid substitution may include the use of non-naturally occurring analogues. The protein or polypeptide used in the present application may also have deletion, insertion or substitution of amino acid residues, where the amino acid residues undergo silent changes and result in functionally equivalent proteins. Intentional amino acid substitutions can be made based on the similarity of the polarity, charge, solubility, hydrophobicity, hydrophilicity, and/or the amphoteric properties of the residues, as long as the endogenous function is retained. For example, negatively charged amino acids include aspartic acid and glutamic acid; positively charged amino acids include lysine and arginine; and amino acids containing uncharged polar head-groups with a similar hydrophilic value include asparagine, glutamine, serine, threonine and tyrosine.

In the present application, the term “homologue” generally refers to an amino acid sequence or a nucleotide sequence having a certain homology with a wild-type amino acid sequence and a wild-type nucleotide sequence. The term “homology” may be equivalent to the “identity” of sequences. Homologous sequences may include amino acid sequences that are at least 80%, 85%, 90%, 99.1%, 99.2%, 99.3%, 99.4%, 99.5%, 99.6%, 99.7%, 99.8% or 99.9% the same as the subject sequence. In general, homologues will contain the same active sites as the subject amino acid sequence, and the like. Homology may be considered on the basis of similarity (i.e., amino acid residues having similar chemical properties/functions), or homology can be expressed in terms of the sequence identity. In the present application, a sequence having a percentage identity in either of the SEQ ID NOs of the mentioned amino acid sequence or nucleotide sequence refers to a sequence having the percentage identity over the whole length of the mentioned SEQ ID NOs. In order to determine the sequence identity, alignment of sequences can be performed by a variety of ways known to those skilled in the art, for example, by using BLAST, BLAST-2, ALIGN, NEEDLE or Megalign (DNASTAR) software, etc. The persons skilled in the art are able to determine the suitable parameters suitable for alignment, including any algorithms required to achieve an optimal alignment in the full-length sequence being compared.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

In the present application, the term “comprising” usually means including, containing, having or encompassing. In some cases, it also refers to the meaning of “being” or “consisting of”.

In the present application, the term “does not comprise” generally refers to the exclusion of the possibility of a certain behavior, structure or structure. For example, “A does not comprise B” generally means to exclude the possibility of B occurring in A.

### TRIM72 Truncated Protein

In one aspect, the present application provides a TRIM72 truncated protein comprising the PRYSPRY domain or its functional fragment. For example, the TRIM72 truncated protein may comprises amino acid sites of 278aa-470aa of the TRIM72 protein. For example, the TRIM72 truncated protein may comprise an amino acid sequence as set for in SEQ ID NO: 2.

In the present application, the TRIM72 truncated protein may further comprise other domain of TRIM72 protein.

For example, the TRIM72 truncated protein may comprise the PRYSPRY domain and coiled-coil domain. For example, the TRIM72 protein may comprise an amino acid sequence as set forth in SEQ ID NO: 11. For example, the TRIM 72 truncated protein may comprise a deletion of B-box domain and Ring-finger domain of TRIM72 protein. For example, the TRIM72 protein may comprise an amino acid sequence as set forth in SEQ ID NO: 11.

Exosomes are small extracellular biological vesicles released into surrounding body fluids through fusion of multivesicular bodies and the plasma membrane, which contain proteins, nucleic acids, lipids and other bioactive substances. For example, the TRIM72 truncated protein may be secreted through exosome. For example, the TRIM72 truncated protein may be packaged in exosome. For example, the exosome may comprise a nucleic acid encoding the TRIM 72 truncated protein. For example, the exosome may be marked by TSG101. When using exosome to secrete the TRIM72 truncated protein, a coiled-coil domain and PRYSPRY domain are needed.

For example, the TRIM72 truncated protein may comprise the PRYSPRY domain and B-box domain. For example, the TRIM 72 truncated protein may comprise a deletion of coiled-coil domain and Ring-finger domain of TRIM72 protein.

For example, the TRIM72 truncated protein may comprise the PRYSPRY domain and the Ring-finger domain. For example, the TRIM 72 truncated protein may comprise a deletion of B-box domain and coiled-coil domain of TRIM72 protein.

For example, the TRIM72 truncated protein may comprise the PRYSPRY domain, the coiled-coil domain and the Ring-finger domain. For example, the TRIM 72 truncated protein may comprise a deletion of B-box domain of TRIM72 protein.

For example, the TRIM72 truncated protein may comprise the PRYSPRY domain, the coiled-coil domain and the B-box domain. For example, the TRIM 72 truncated protein may comprise a deletion of Ring-finger domain of TRIM72 protein.

For example, the TRIM72 truncated protein may comprise the PRYSPRY domain, the Ring-finger domain and the B-box domain. For example, the TRIM 72 truncated protein may comprise a deletion of coiled-coil domain of TRIM72 protein.

In the present application, the TRIM72 truncated protein may comprise its variants. For example, the TRIM72 truncated protein may comprise one or more amino acid mutations compared with the correspondence wild type sequence. For example, the TRIM72 truncated protein may comprise amino acid sequence that are at least 80%, 85%, 90%, 99.1%, 99.2%, 99.3%, 99.4%, 99.5%, 99.6%, 99.7%, 99.8% or 99.9% the same as the correspondence wild type sequence.

For example, the TRIM72 truncated protein may comprises an amino acid sequence as set forth in SEQ ID NO: 6, 7, 8, 9, or 11, or an amin acid sequence with at least 80%, 85%, 90%, 99.1%, 99.2%, 99.3%, 99.4%, 99.5%, 99.6%, 99.7%, 99.8% or 99.9% similarity with SEQ ID NO: 6, 7, 8, 9, or 11.

In the present application, the TRIM72 truncated protein may comprise an amino acid mutation at position C14. For example, the amino acid mutation may be C14A.

According to applicant's research, amino acid cysteine at position 242 is critical for oligomer formation of TRIM72 protein. Substitution of amino acid C242 may block the protection function of neurons. Therefore, amino acid substitution at position C242 (for example, C242A) may not be included in the TRIM72 truncated protein in the present application.

In the present application, the truncated TRIM72 protein may be used for protect neurons by reducing oxidative stress. In the present application, the truncated TRIM72 protein may be used for preventing or treating a nervous system disease associated with oxidative stress, for example, the nerve damage disease or neurodegenerative disease.

In the present application, the truncated TRIM72 protein may be used for preventing or treating a nervous system disease. For example, the present application may be used for preventing and/or treating ALS, PD, and/or Stroke.

### Recombinant Protein, Nucleic Acid, Vector, Cell, Fusion Protein, Pharmaceutical Composition

In another aspect, the present application provides a recombinant protein comprising the TRIM72 truncated protein.

In another aspect, the present application provides one or more nucleic acid molecules capable of encoding the TRIM72 truncated protein of the present application.

In some embodiments, said nucleic acid molecule can completely encode the TRIM72 truncated protein or fusion protein of the present application. For example, said TRIM72 truncated protein or fusion protein can be obtained by use of only one type of nucleic acid molecule. In some embodiments, said nucleic acid molecule can encode a part of the TRIM72 truncated protein or fusion protein of the present application. For example, said fusion protein can be obtained by use of more than two types of different said nucleic acid molecules.

In another aspect, the present application provides one or more vectors which can comprise one or more nucleic acid molecules of the present application. In another aspect, the present application provides a cell (e.g., a host cell), which can comprise the nucleic acid molecule of the present application or the vector of the present application.

In the present application, the vector can be a polynucleotide that can be transcribed and translated into a polypeptide when introduced into a suitable host cell. Generally, by culturing a suitable host cell containing said vector, said vector can produce the desired expression product. In the present application, said vector can include one or more of said nucleic acid molecules. For example, said vector can comprise all the nucleic acid molecules required for encoding said TRIM72 truncated protein.

In addition, said vector can also include other genes, such as a marker gene that allows selecting the vector in a suitable host cell and under suitable conditions. In addition, said vector can also include an expression control element that allows the coding region to be properly expressed in a suitable host. Such control element is well known to those skilled in the art. For example, they can comprise promoters, ribosome binding sites, enhancers, and other control elements that regulate gene transcription or mRNA translation. In some embodiments, said expression control sequence is a regulatory element. The specific structure of said expression control sequence can vary depending on the function of the species or cell types, but usually comprises 5′ non-transcribed sequences and 5′ and 3′ non-translated sequences involved in transcription and translation initiation, such as TATA boxes, capped sequences, CAAT sequences, etc. For example, the 5′ non-transcribed expression control sequence can comprise a promoter region, and the promoter region can comprise a promoter sequence for transcriptional control of the functionally linked nucleic acid.

In another aspect, the present application provides a pharmaceutical composition, comprising said TRIM72 truncated protein, the recombinant protein, the nucleic acid molecule, the vector, the cell and/or the fusion protein, and a pharmaceutically accepted adjuvant.

In some embodiments, pharmaceutically acceptable adjuvant can comprise buffers, antioxidants, preservatives, low molecular weight polypeptides, proteins, hydrophilic polymers, amino acids, sugars, chelating agents, counter-ions, metal complexes, and/or nonionic surfactants etc.

In some embodiments, the pharmaceutically accepted adjuvant can comprise drug, toxins, cytokines, radioactive elements, carrier proteins, enzymes, lectins, fluorescent quantum dots, and/or high absorption coefficient of chromophore.

In the present application, said pharmaceutical composition can be formulated with a pharmaceutically acceptable carrier or diluent and any other known adjuvants and excipients according to conventional technical means in the art, e.g., following the operations in Remington: The Science and Practice of Pharmacy, nineteenth edition, edited by Gennaro, Mack Publishing Co., Easton, PA, 1995.

In the present application, said composition can be formulated for oral administration, intravenous administration, intramuscular administration, in situ administration at the tumor site, inhalation, rectal administration, vaginal administration, transdermal administration or the medicine is administered via a subcutaneous depot.

In the present application, said pharmaceutical composition can be used to protect neurons. For example, the composition of the present application can inhibit or delay the development or progression of nervous system diseases (e.g., ALS, PD, or Stroke), and/or can reduce and/or stabilize the disease status.

The pharmaceutical composition of the present application can comprise a therapeutically effective amount of said TRIM72 truncated protein. Said therapeutically effective amount is a dose required to prevent and/or treat (at least partially treat) diseases (e.g., ALS, PD, or Stroke) and/or any complications thereof in a subject with or at a risk of the diseases.

### Preparation, Method and Use

In another aspect, the present application provides a method for protecting neurons in a subject, comprising administering an effective amount of the TRIM72 truncated protein, the recombinant protein, the nucleic acid molecule, the vector, the cell, the fusion protein, and/or the pharmaceutical composition to a subject in need thereof.

In another aspect, the present application provides a method for preventing and/or treating a nervous system disease, comprising administering an effective amount of the TRIM72 truncated protein, the recombinant protein, the nucleic acid molecule, the vector, the cell, the fusion protein, and/or the pharmaceutical composition to a subject in need thereof.

In another aspect, the present application provides a use of the TRIM72 truncated protein, the recombinant protein, the nucleic acid molecule, the vector, the cell, the fusion protein, and/or the pharmaceutical composition in manufacture of a drug for preventing and/or treating a nervous system disease.

In another aspect, the present application provides the TRIM72 truncated protein, the recombinant protein, the nucleic acid molecule, the vector, the cell, the fusion protein, and/or the pharmaceutical composition, for use in preventing and/or treating a nervous system disease.

In the present application, the nervous system disease comprises ALS.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); rpm, revolutions per minute; and the like.

### Methods

Preparation of Recombinant TRIM72 Proteins in E. coli

Codon optimization and gene synthesis were performed based on the amino acid sequence (Sequence1) of mouse TRIM72. TRIM72 full-length and truncations were constructed in pET21b vector with 6×His tag in the N-terminus. These proteins were expressed and purified from E. coli BL21 (DE3) cells (Millipore) and purified under native conditions unless otherwise noted. E. coli were grown to OD600 of 0.8 and induced with 0.6 mM IPTG at 16 degree overnight. Pelleted cells were resuspended in lysis buffer (250 mM NaCl, 50 mM HEPES 7.5, 1 mM DTT, protease inhibitor). After sonication, lysates were pelleted at 30,000×g at 4° C. for 30 min. Supernatants were applied to Ni columns with 10 mL beads (GE) prewashed with lysis buffer at room temperature. Proteins were eluted with elution buffer (25 mmol/L Tris pH 8.0, 300 mmol/L NaCl, 200 mmol/L imidazole). The proteins were further treated with 0.1 mg/ml RNaseA (Thermo Fisher) to remove RNA, and then purified by Superdex 200 16/200 column (GE) equilibrated in SEC buffer (400 mM NaCl, 50 mM HEPES 7.5, 1 mM DTT). The fractions were analyzed by SDS-PAGE, pooled, concentrated, filtered, flash frozen in liquid nitrogen, and stored at −80° C.

### Preparation of Recombinant TRIM72 Protein and It's Truncated Fragments

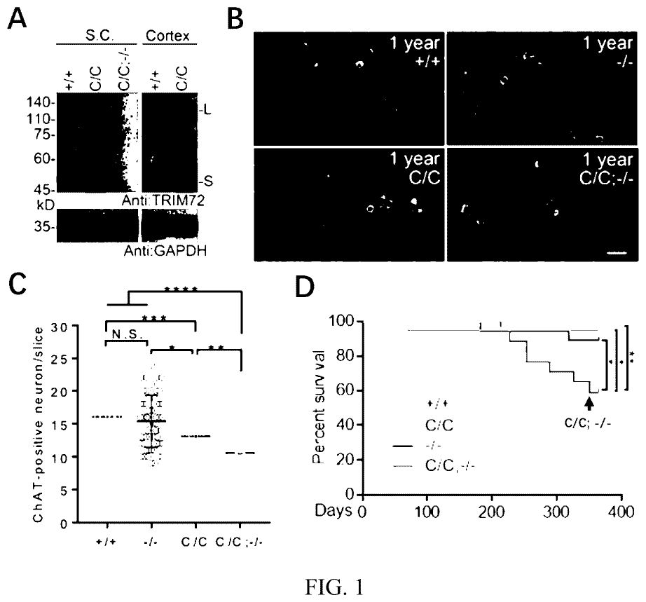

Codon optimization and gene synthesis were performed based on the amino acid sequence (SEQ ID NO: 2) of human TRIM72. With 6×His tag added to the amino terminus, the synthetic TRIM72 gene were used as a template to construct pcDNA3.1-6His-TRIM72, pcDNA3.1-6His-coiled-coil, pcDNA3.1-6His-PRYSPRY and pcDNA3.1-6His-coiled-PRYSPRY plasmids through gene amplification, respectively (FIG. 1). High quality endotoxin-free plasmids were obtained by plasmid amplification using E. coli. HEK293F cells were transfected, collected and lysed, and protein lysates were collected. TRIM72 (full-length) protein and its truncated fragments (Coiled-coil (SEQ ID NO: 5), PRYSPRY (SEQ ID NO: 6), and Coiled-PRYSPRY (SEQ ID NO: 11)) were prepared after purification of lysates by Ni-affinity column.

### Plasmids and Lentiviral Vectors

DNA fragments corresponding to full-length of TRIM72 were amplified from a mouse cDNA library by PCR and inserted into pCMV-N-3×Flag expression vector between SalI and XhoI sites using seamless Cloning kit (Beyotime) to generate Flag-tagged TRIM72. The series of Flag-tagged TRIM72 mutants: C14A (the cysteine at position 14 substituted by alanine); C242A (the cysteine at position 242 substituted by alanine) were generated from the wild-type TRIM72 construct by point mutation. The series of Flag-tagged TRIM72 domain deletion: ΔRing domain (deletion of 14-69 domain, SEQ ID NO: 7); ΔB-box domain (deletion of 81-122aa, SEQ ID NO: 8); Δcoiled-coil domain (deletion of 135-232aa, SEQ ID NO: 9); ΔPRYSPRY domain (deletion of 278-470aa, SEQ ID NO: 10) construct was generated from the full-length of TRIM72 expression vector. Flag-tagged TRIM72 coiled-coil domain (135-232aa) or PRYSPRY domain (278-470aa) or coiled-coil domain plus PRYSPRY domain (135-470aa) construct was generated from the full-length TRIM72 expression vector.

For TRIM72 stable expression cell line construction, DNA fragments of the series of Flag-tagged TRIM72 mutants or domain deletion or single domain were amplified from the corresponding TRIM72 expression vector by PCR and inserted into pLJM1-EGFP lentiviral vector between BsrGI and EcoRI sites to generate the series of Flag-tagged TRIM72 mutants or domain deletion or single domain fused with EGFP in lentiviral vector.

Lentivirus expressing Flag-tagged TRIM72 construct was generated from the corresponding EGFP fused with Flag-tagged TRIM72 vector.

### Cell Culture, Lentiviral Packaging and Lentiviral Infection

HEK293FT cells were maintained in DMEM (Invitrogen) with 10% fetal bovine serum (Gemini) in cell incubator (37° C., 5% CO₂). For lentiviral packing, HEK293FT cells were seeded in growth medium for three 10-cm culture dish. When reaching around 90% confluence, cells were co-transfected with VSVG (10 μg), pxPAX2 (15 μg) and pLJM1-EGFP lentiviral vector or pLentiCRISPRv2 (Addgene) or pLenticas9-Blast (Addgene) (20 μg) using PEI (Sigma) following manufacturer's instructions and changed medium with fresh growth medium 5-6 h after transfection. The medium was harvested 72 hours after transfection and centrifugated at 20,000 rpm, 4° C. for 2 hours. After centrifugation, the lentivirus was enriched in the pellet. The lentivirus was resuspended using 100 μl DPBS and stored in −80° C.

HEK293FT cells or Hela cells were infected with indicated lentivirus. After 3 days of infection, the infected cells were selected with 2 μg/ml puromycin or 10 μg/ml blasticidin according to the plasmid containing resistance for at least a week. The puromycin- or blasticidin-selected cells were applied for further analysis.

### Cell Viability Assay

Cell viability was assessed using CCK-8. Cells were seeded in a 96-well plate at a density of 1.5×10³ cells per well for Arsenite treatment or 7×10³ cells per well for H₂O₂ treatment. Arsenite (Sigma) was added into each well at concentration of 0.125 mM, 0.25 mM or 0.5 mM, and washed the cells after 2 hours incubation at 37° C. For H₂O₂-treatment experiment, scAAV9 infection (estimated multiplicity of infection (MOI): 10,000 vg/cell) was done for 24 hours at 17 hours after cell seeding. Then, H₂O₂ was added into each well at concentration of 300 μM, and washed the cells after 1 hours incubation at 37° C. After cells have been processed by Arsenite or H₂O₂. a total of 10 μL of CCK-8 solution (Yeasen) was added to each well. After another 2 hours of incubation at 37° C., the optical density (OD) value of each well was measured using a microplate reader with an excitation wavelength of 450 nm. The cell viability of 293 FT was calculated. The experiment was repeated at least three times to obtain the mean value.

### Exosome Purification

The protocol for purifying exosomes from 100 ml supernatant of 293 FT cells included two steps of ultrafiltration and polyethylene glycol (PEG) precipitation. First, pour the collected supernatant into a centrifuge tube and centrifuge at 3000×g for 20 minutes to remove cell debris. Then, filter the supernatant once with a 0.45 μm filter. After that, take a new Amicon ULTRA-15 ultrafiltration tube washed with PBS or autoclaved water. Then immediately add the supernatant to the ultrafiltration tube, and centrifuge at 3000×g for 5-10 minutes. Discard the filtrate, continue to add the supernatant, and centrifuge at 3000×g for 5-10 minutes until all the supernatant is introduced into the ultrafiltration tube. Then transfer the concentrate to a 50 mL centrifuge tube. Add isolation reagent to the concentrate and mix samples thoroughly by vortexing or pipetting. Finally, leave the samples at 2-8° C. overnight. The next day, samples were centrifuged at 10,000×g for 1 hour at 4° C. Discard the supernatant, the exosomes were in the pellet.

### Western Blot Analysis

Total protein content in tissues and cells was extracted using RIPA lysis buffer (50 mM Tris-HCl, pH 8.0, 150 mM NaCl, 1 mM EDTA, 0.1% SDS, 1% TritonX-100, 0.5% Sodium deoxycholate) supplemented with phenymethanesulfonyl fluoride (PMSF) and proteinase inhibitor cocktail (Bimake) and the lysate was incubated for 30 minutes on ice. After 12000 rpm centrifugation for 10 minutes, the supernatant was extracted and was incubated at 95° C. for 10 minutes after mixing with SDS loading buffer. Next, the proteins were separated by 10% of sodium dodecyl sulfate-polyacrylamide gel electrophoresis (SDS-PAGE) and transferred onto a polyvinylidene fluoride (PVDF) membrane. Afterward, the membranes were blocked using 5% nonfat milk for 1 hours at room temperature. Then incubated with diluted primary antibodies overnight at 4° C., including GAPDH (Ameribio) TUBULIN (Ameribio), TRIM72 antibody (a kindly gift from Dr. Jianjie Ma), CCDC22 (Sigma), GFP (Abcam), Myc (Abmart), TSG101 (Abcam), ITGAV (Abcam), H₃ (Abcam) and Flag (Abmart). And then the membranes were incubated with HRP-conjugated secondary antibodies at room temperature for 1 hour. Images were analyzed using the Fiji ImageJ to obtain the integrated intensities.

### Arsenite Intragastric Administration

Arsenite was prepared as 1 mg/ml in ddH₂O and then injected i.g. as 6 mg/kg body weight into mice as described in experiment (Zhang et al., 2020).

### ROS Measurement

ROS level was measured using dihydroethidium (DHE, Invitrogen) and MitoSOX (Invitrogen). The cortical neurons were plated on 24-well chamber slides and cultured to DIV12-14. And then the neurons were incubated with DHE (20 μM in medium) or MitoSOX (5 μM in medium) at 37° C. for 20 min. After incubation, the neurons were fixed with 4% PFA at room temperature for 10 min. Images were captured by Nikon A1 confocal microscope. DHE fluorescence was excited at 535 nm and collected the emission at 610 nm. MitoSOX fluorescence was excited at 510 nm and collected the emission at 580 nm. The mean intensity of DHE or MitoSOX fluorescence were accessed by FiJi ImageJ software. And the ROS level was expressed by the relative mean intensity of DHE or MitoSOX fluorescence in different groups. The RMI=mean intensity of DHE or MitoSOX fluorescence divided by mean intensity of background.

For lentiviral infection, cortical neurons were plated on 24-well chamber slides. And cultured to DIV3-5, every well was added 10 μl purified lentivirus for further culture. When cultured to DIV12-14, the neurons were performed DHE staining.

### Cortical Neuron Culture

The cortices were dissected from postnatal day 0 mouse pups in HBSS (Invitrogen) and incubated with trypsin in Neurobasal™ (Invitrogen) at 37° C. for 15 min. During the digestion, DNAse I (Sigma, 10 μg/ml) was added in the last 10 min. Stop the trypsinization process by adding Neurobasal medium with 10% FBS. After digestion, dissociate the tissue by gentle titration to obtain a single cell suspension. The suspension was passed through a 70 μm strainer. After centrifugation at 800 g for 10 min, the cells were resuspended in Neurobal™ with 10% FBS, GlutaMAX™ (Invitrogen) and plated in 24-well plate at the density of 2×10⁵ cells/ml at 500 ml per well. After 5-6 h incubation (5% CO₂, 37° C.), replaced the medium with Neurobal™ with B27 (Invitrogen), GlutaMAX™ (Invitrogen). The cultured neurons (12-14 days in vitro) were stained by DHE or MitoSOX.

### Example 1 TRIM72 Knockout Accelerates Disease Progression Shown in FUS-R521C KI Mouse

TRIM72, a protein with undetectable level in central nervous system, is significantly upregulated in FUS-R521C KI mice (FIG. 1A). To examine the biological consequences of TRIM72 up-regulation in FUS-R521C KI mice, we analyzed the motor neurons and lifetime in wildtype (+/+), FUS-R521C (C/C), FUS-R521C with TRIM72 knockout (C/C;−/−), and TRIM72 knockout alone (−/−) animals. A significant more motor neuron loss in spinal cord was observed in C/C;−/− animals compared to C/C mutants at the age of one year (FIG. 1B, C). However, the TRIM72 knockout mice did not display the motor neuron loss compared to +/+ controls (FIG. 1C). The survival curve of these four genotypes agreed that C/C;−/− animals have shorter lifespan (˜40% of C/C;−/− animals died at one year of age) compared to other three genotypes (FIG. 1D).

### Example 2 TRIM72 Knockout Increases Oxidative Stress in FUS-R521C KI Mouse

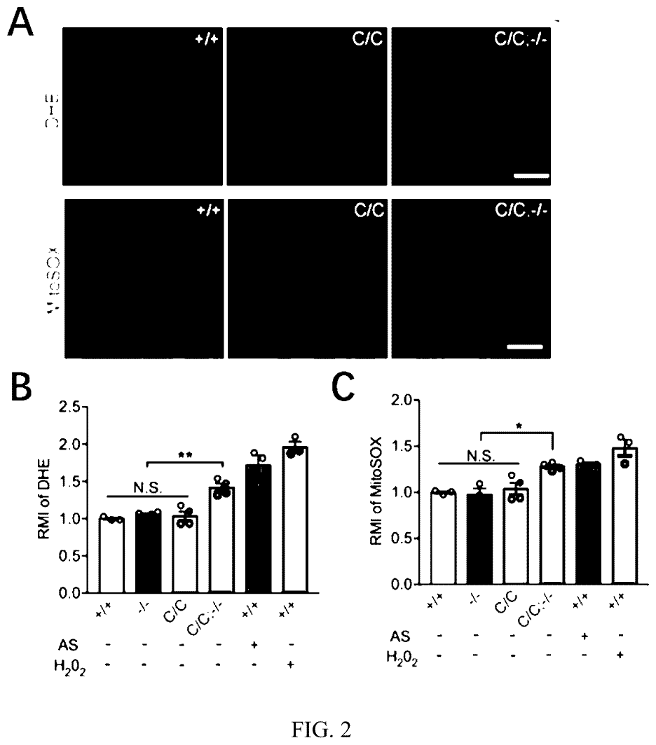

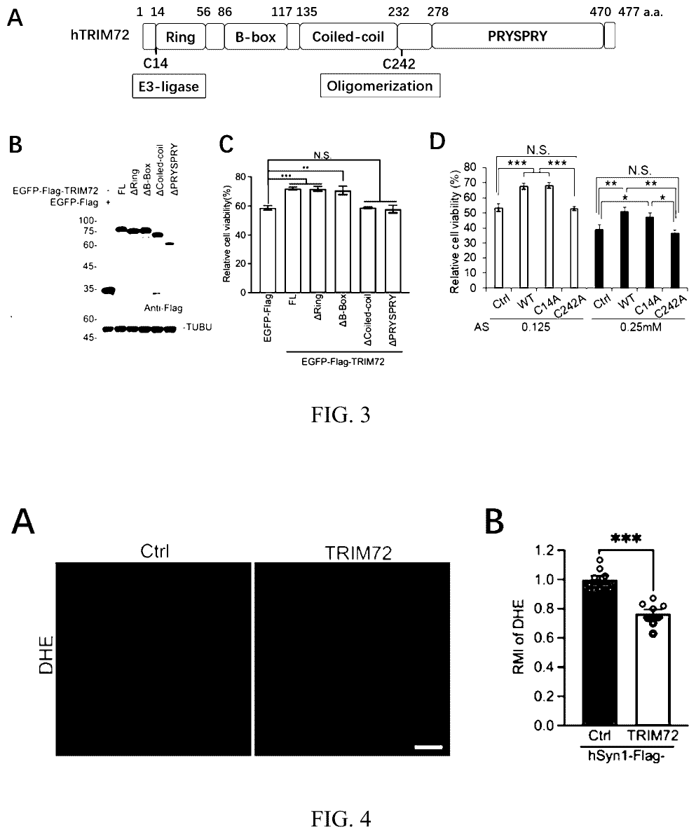

To study the mechanism how loss-of-function of TRIM72 accelerates the disease progression of FUS-R521C KI mice, we cultured cortical neurons from these four genotype animals and measured ROS by Dihydroethidium (DHE) and MitoSOX staining, both of which are superoxide indicators. As positive controls, wildtype cultures treated with arsenite (AS, an oxidative stress inducer) and H₂O₂ displayed higher relative mean intensity (RMI) of DHE and MitoSOX staining (FIG. 2A-C). Although the RMI values were comparable between +/+, −/− and C/C cortical neurons, the values were significantly higher in cortical neurons from C/C;−/− animals (FIG. 4G, 4H), indicating that loss-of-function of TRIM72 indeed increases the ROS accumulation in FUS-R521C mice.

### Example 3 Overexpression of TRIM72 Protects Cells from Oxidative Stress

TRIM72 protein contains Ring finger motif, B-box domain, coiled-coil domain and PRYSPRY domain (FIG. 3A). To investigate which domain of TRIM72 plays a role in protection from oxidative stress, full-length TRIM72 or domain-disrupted TRIM72 mutants overexpressed 293 FT cell line was constructed by lentiviral infection and puromycin-selection (FIG. 3B). CCK-8 was used to measure the cell viability after arsenite (AS, an oxidative stress inducer) treatment. Indeed, a decrease in cell viability with AS treatment and overexpression of TRIM72 increased the cell viability (FIG. 3C) was observed. It was found that coiled-coil domain- and PRYSPRY domain-disrupted TRIM72 mutants abolished the protective effect of TRIM72, while Ring domain- or B-box domain-disrupted TRIM72 mutants reserved equal protective effect to wildtype (FIG. 3C).

Previous study determined that TRIM72 senses changes in the oxidative environment and forms oligomer complex to complete membrane repair. A cystidine residue (C242) play a critical role in TRIM72 oligomer formation. In addition, the cystidine residue (C14) is critical for TRIM72 E3 ligase activity. Mutation of C424 into alanine (C242A) blocked TRIM72 protective effect, while its E3 ligase inactive mutant (C14A) reserved equal protective effect to wildtype (FIG. 3D). Therefore, it is concluded that TRIM72 protects cells from oxidative stress and is mainly dependent on its oligomerization not E3 ligase activity.

To study whether TRIM72 protects neurons from oxidative stress, lentivirus was used to restore TRIM72 expression in C/C;−/− cortical neurons. More than 95% neurons expressed TRIM72 and a reduction of DHE staining in TRIM72 expressed neurons could be observed (FIG. 4A, B).

### Example 4 Recombinant TRIM72 Proteins Protects Cortical Neurons Against Oxidative Stress

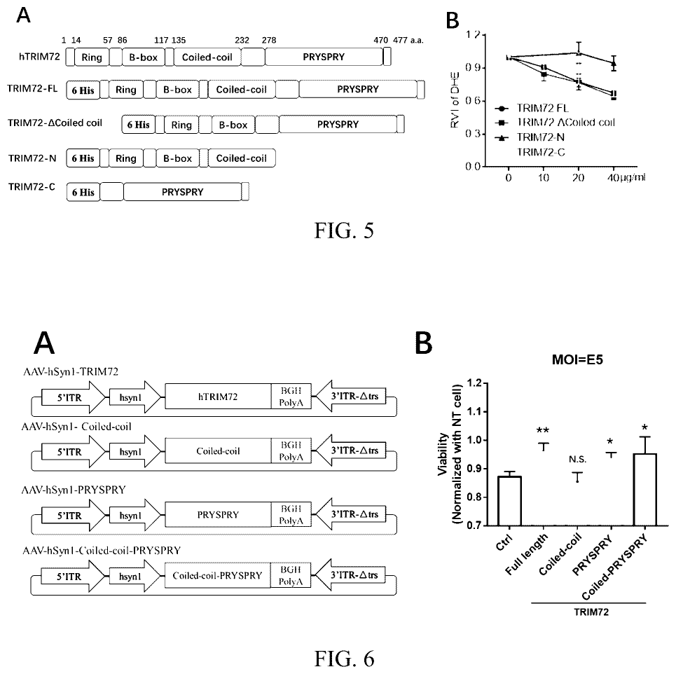

Next, to examine whether recombinant TRIM72 protein can elicit the survival function in cultures, TRIM72 proteins purified from E. coli was used. We found that TRIM72 at more than 20 μg/ml could significantly decrease ROS level and exhibited a dose-dependent effect in cultured cortical neurons (FIG. 5). Further, it was also demonstrated that C-terminal but not N-terminal fragment of TRIM protein mediated the effects of ROS reduction (FIG. 5), which means that PRYSPRY single domain exhibited the endogenous antioxidant capacity, could significantly decrease ROS level and increase the cell viability. Deletion of coiled-coil domain from TRIM72 protein did not abolish the protective effect, which is different from the results of gene expression in FIG. 3C.

### Example 5 AAV-TRIM72 Packaging and scAAV-TRIM72 Protects N2a Cells from Oxidative Stress

AAV packaging system is a commonly used triple-plasmid system. By simultaneously transfecting the three plasmids into mammalian cells (e.g. HEK293), all components required for AAV packaging can be expressed and assembled into virus particles in this cell. Here, we used a modified triple-plasmid system from PackGene (Guangzhou PackGene Biotech Co., Ltd). This system consists of three plasmids: pAAV-ITR containing target gene, serotype vector pRepCapX and helper vector pADHelper. The target vector pAAV-ITR contains eukaryotic promoters and other components required for high levels of gene expression in mammalian cells when foreign sequences are cloned into polyclonal sites (MCS). The vector also contains AAV reverse terminal repeat sequences (ITRs) that guide virus replication and packaging. Vector pRepCapX contains AAV rep and CAP genes that encode replication proteins and viral capsid proteins. Stabilization of rep and CAP gene expression levels is a key step in obtaining desired high titer viral products. Vector pADHelper contains a collection of adenovirus genes VA, E2A, and E4 that are essential for cell production of high-titer viruses. FIG. 6 showed the construction of pAAV-ITR vectors for scAAVs.

To further investigate which key domains of TRIM72 protein are necessary to protect neurons, different domain-deleted TRIM72 were constructed and overexpressed in N2a cell line by scAAV9 infection (FIG. 6A). CCK-8 was used to measure the cell viability after H₂O₂ treatment. Indeed, cell viability was decreased after H₂O₂ treatment (FIG. 6B). It is noteworthy that overexpression of Coiled-coil-PRYSPRY or the PRYSPRY single domain but not the coiled-coil single domain was sufficient to protect cells from oxidative stress, suggesting that either the Coiled-coil-PRYSPRY construct or the PRYSPRY only could elicit protective effect (FIG. 6B).

### Example 6. TRIM72 can be Secreted Through Exosome

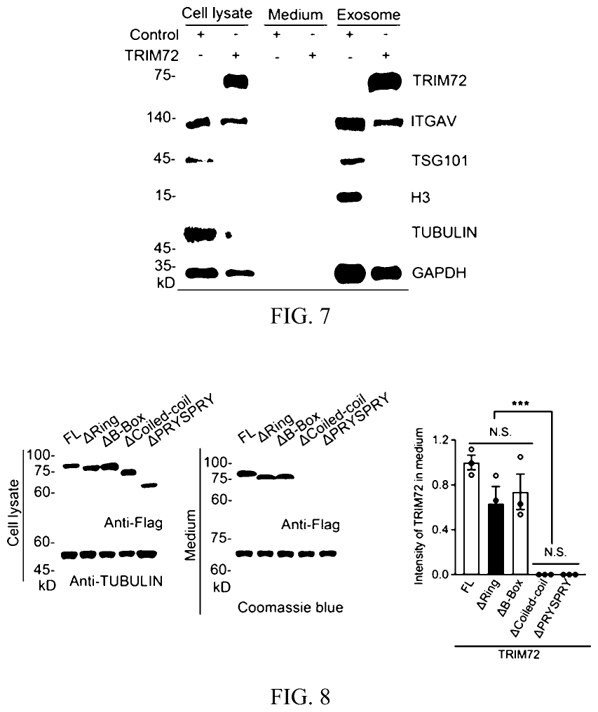

Exosomes are small extracellular biological vesicles released into surrounding body fluids through fusion of multivesicular bodies and the plasma membrane, which contain proteins, nucleic acids, lipids and other bioactive substances. Exosomes play an important role in the exchange of information between cells by releasing bioactive substances that fuse with receptor cell membranes or bind to cell surface receptors. Full-length TRIM72 or different domain-disrupted TRIM72 mutants were constructed and stably overexpressed in 293 FT cell line by lentiviral infection. We found that TRIM72 is enriched in TSG101-labeled exosomes, which means that TRIM72 could affect the biological processes of surrounding cells through the exosomal secretion pathway (FIG. 7). Further analysis showed that Coiled-coil domain and PRYSPRY domain are necessary for their exosome secretion, and the deletion of either domain will abolish the secretion of TRIM72 through exosomes (FIG. 6). Comparatively, removing the ring domain or B-box domain only has a limited impact on the secretion efficiency of TRIM72 (FIG. 8).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A TRIM72 truncated protein, comprising the PRYSPRY domain or its functional fragment of a TRIM72 protein.

2. The TRIM72 truncated protein of claim 1, wherein said TRIM72 protein is a human TRIM72 protein.

3. (canceled)

4. (canceled)

5. The TRIM72 truncated protein of claim 1, wherein said PRYSPRY domain comprises an amino acid sequence as set forth in SEQ ID NO: 6.

6. The TRIM72 truncated protein of claim 1, further comprising the coiled-coil domain or its functional fragment of a TRIM72 protein.

7. (canceled)

8. (canceled)

9. The TRIM72 truncated protein of claim 6, wherein said coiled-coil domain comprises an amino acid sequence as set forth in SEQ ID NO: 5.

10. The TRIM72 truncated protein of claim 1, further comprising the B-box domain or its functional fragment of a TRIM 72 protein.

11. (canceled)

12. (canceled)

13. The TRIM72 truncated protein of claim 10, wherein said B-box domain comprises an amino acid sequence as set forth in SEQ ID NO: 4.

14. The TRIM72 truncated protein of claim 1, further comprising the Ring-finger domain or its functional fragment of a TRIM 72 protein.

15. (canceled)

16. (canceled)

17. The TRIM72 truncated protein of claim 1, wherein said Ring-finger domain comprises an amino acid sequence as set forth in SEQ ID NO: 3.

18. The TRIM72 truncated protein of claim 1, comprising the amino acid sequence as set forth in any one of SEQ ID NO: 6, 7, 8, 9 and or 11.

19. The TRIM72 truncated protein of claim 1, comprising its variant thereof.

20-23. (canceled)

24. The TRIM72 truncated protein of claim 1, is secreted through exosome.

25-27. (canceled)

28. A recombinant protein, comprising said TRIM72 truncated protein of claim 1.

29. One or more isolated nucleic acid molecules, encoding said TRIM72 truncated protein of claim 1.

30. A vector, comprising said nucleic acid molecule of claim 29.

31. (canceled)

32. The vector of claim 30, comprising an AAV vector.

33. (canceled)

34. (canceled)

35. A fusion protein, comprising said TRIM72 truncated protein of claim 1.

36. A pharmaceutical composition, comprising said TRIM72 truncated protein of claim 1, and a pharmaceutically accepted adjuvant.

37. (canceled)

38. (canceled)

39. A method for preventing and/or treating a nervous system disease, comprising administering an effective amount of said TRIM72 truncated protein of claim 1 to a subject in need thereof.

40. (canceled)

41. (canceled)

42. The method of claim 39, wherein said nervous system disease comprising ALS, PD, and/or ALS.

43-46. (canceled)
